(19) United States
(12) Patent Application Publication
(10) Pub. No.: US 2026/0092068 A1
(43) Pub. Date: Apr. 2, 2026

(54) PHYSICAL FORMS OF AN INHIBITOR OF PRC2

(71) Applicant: ORIC Pharmaceuticals, Inc., South San Francisco, CA (US)

(72) Inventors: Mohammad AL-SAYAH, South San Francisco, CA (US); Robert Ronald MILBURN, South San Francisco, CA (US)

(21) Appl. No.: 19/110,340

(22) PCT Filed: Sep. 13, 2023
(86) PCT No.: PCT/US2023/074029

§ 371 Date: Mar. 10, 2025

Related U.S. Application Data
(60) Provisional application No. 63/375,582, filed on Sep. 14, 2022.

(51) Int. Cl. C07D 487/04

(52) U.S. Cl. CPC C07D 487/04

(57) ABSTRACT

The present disclosure relates to physical forms of a PRC2 inhibitor compound, pharmaceutical compositions comprising the physical forms of the compound, and methods of treating subjects having cancer by administering the physical forms, or a pharmaceutical composition comprising one or more of the physical forms, to the subject.

### CROSS-REFERENCE

This application claims the benefit of U.S. Provisional Application Ser. No. 63/375,582 filed Sep. 14, 2022; which is hereby incorporated by reference in its entirety.

### BACKGROUND OF THE INVENTION

The Polycomb Repressive Complex 2 (PRC2) is a multiprotein complex that contributes to the epigenetic silencing of target genes to regulate development and homeostasis. The PRC2 complex is comprised of three core subunits: enhancer of zeste homolog 2 (EZH2), embryonic ectoderm development protein (EED), and suppressor of zeste 12 (SUZ12). Two additional non-essential subunits, AEBP2, and RbAp48, function to promote the enzymatic activity of the PRC2 complex (e.g., see Cao et al., (2002) Science 298: 1039-1043). The dysregulation of PRC2 methyltransferase activity can lead to tumorigenesis in a wide range of cancers including prostate cancer, breast cancer, skin cancer, bladder cancer, liver cancer, pancreatic cancer, head and neck cancer, and hematological malignancies. PRC2 is composed of two druggable subunits: EED and EZH2. The pharmacologic properties of some EZH2 inhibitor compounds may require high doses that achieve only partial target inhibition in subjects suffering from cancer. Additionally, preclinical studies suggest drug resistance to EZH2 inhibitors may develop via EZH1 bypass compensation or acquired mutations in EZH2. Allosteric inhibition of EED impacts the assembly, stabilization, and activation of PRC2, and may have benefits over EZH2-mediated inhibition of PRC2. Therefore, there is a need to develop compounds, and pharmaceutically acceptable compositions comprising the compounds, that exhibit inhibition of the EED subunit of PRC2 for the treatment of cancer in subjects.

### SUMMARY OF THE INVENTION

Provided herein are physical forms of Compound 1:

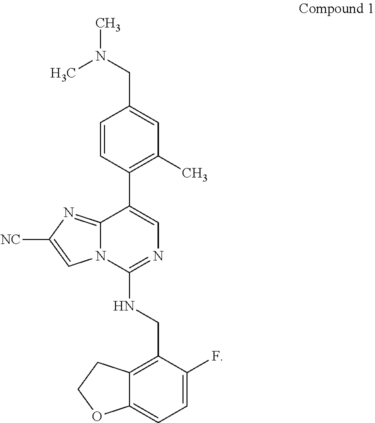 [SMILES: Cc1cc(CN(C)C)ccc1-c1cnc(NCc2c(F)ccc3c2CCO3)n2cc(C#N)nc12]

Also provided herein are crystalline forms of Compound 1. Further provided herein are crystalline forms of Compound 1, wherein the crystalline form is an anhydrous form. Also provided herein are pharmaceutical compositions comprising an amount of a crystalline form of Compound las disclosed herein, and at least one pharmaceutically acceptable excipient.

In another embodiment are provided methods of inhibiting PRC2 activity in a subject in need thereof, comprising administering to the subject an effective amount of a solid form of Compound 1, including a crystalline form of Compound 1 as disclosed herein.

In other embodiments are provided methods of treating cancer in a subject, comprising administering to the subject an effective amount of a solid form of Compound 1, including a crystalline form of Compound 1 as disclosed herein. In another embodiment are provided such methods of treating cancer in a subject, wherein the cancer is a PRC2-associated cancer.

### DETAILED DESCRIPTION OF THE INVENTION

As used herein and in the appended claims, the singular forms “a,” “an,” and “the” include plural referents unless the context clearly dictates otherwise. Thus, for example, reference to “an agent” includes a plurality of such agents, and equivalents thereof known to those skilled in the art, and so forth. When ranges are used herein for physical properties, such as molecular weight, melting points, or chemical properties, such as chemical formulae, all combinations and sub-combinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

As used in the specification and appended claims, unless specified to the contrary, the following terms have the meaning indicated below.

“Administering” when used in conjunction with a therapeutic means to administer a therapeutic systemically or locally, as directly into or onto a target tissue, or to administer a therapeutic to a subject whereby the therapeutic positively impacts the tissue to which it is targeted. Thus, as used herein, the term “administering,” when used in conjunction with a composition described herein, can include, but is not limited to, providing a composition into or onto the target tissue; providing a composition systemically to a subject by, e.g., oral administration whereby the therapeutic reaches the target tissue or cells. “Administering” a composition may be accomplished by injection, topical administration, and oral administration or by other methods alone or in combination with other known techniques.

The terms “crystalline” and “crystallinity” refer to a solid composition having some measure of long-range order in the position of its molecules, as measured by analytical techniques known to those having ordinary skill in the art, such as x-ray powder diffraction (XRPD).

The term “differential scanning calorimetry,” as used herein means a method of thermal analysis described in USP <891>.

The term “pharmaceutically acceptable,” means the carrier, diluent or excipient must be compatible with the other ingredients of the formulation and not deleterious to the recipient thereof.

The term “pharmaceutical composition” shall mean a composition comprising one or more active ingredients, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “therapeutic” means an agent utilized to treat, combat, ameliorate, prevent, or improve an unwanted condition or disease of a subject.

A “therapeutically effective amount” or “effective amount” as used herein refers to the amount of active compound or pharmaceutical agent that elicits a biological or medicinal response in a tissue, system, animal, individual or human that is being sought by a researcher, veterinarian, medical doctor or other clinician, which includes one or more of the following: (1) preventing the disease; for example, preventing a disease, condition or disorder in an individual that may be predisposed to the disease, condition or disorder but does not yet experience or display the pathology or symptomatology of the disease, (2) inhibiting the disease; for example, inhibiting a disease, condition or disorder in an individual that is experiencing or displaying the pathology or symptomatology of the disease, condition or disorder (i.e., arresting further development of the pathology and/or symptomatology), and (3) ameliorating the disease; for example, ameliorating a disease, condition or disorder in an individual that is experiencing or displaying the pathology or symptomatology of the disease, condition or disorder (i.e., reversing the pathology and/or symptomatology).

The terms “treat,” “treated,” “treatment,” or “treating” as used herein refers to therapeutic treatment in some embodiments, wherein the object is to prevent or slow (lessen) an undesired physiological condition, disorder, or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

As used herein, the term “x-ray powder diffraction (XRPD)” means the technique of characterizing a solid for crystallinity or partial crystallinity by use of powder x-ray diffraction as set forth in USP <941>.

Provided herein are crystalline forms of Compound 1:

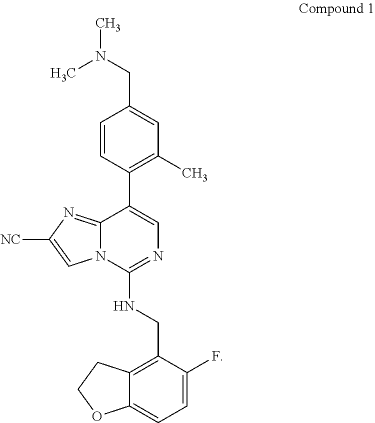 [SMILES: Cc1cc(CN(C)C)ccc1-c1cnc(NCc2c(F)ccc3c2CCO3)n2cc(C#N)nc12]

Also provided herein are crystalline forms of Compound 1, wherein the crystalline forms are anhydrous forms.

Further provided herein is a crystalline form of Compound 1, wherein the crystalline form exhibits a peak in an x-ray powder diffraction (XRPD) pattern at 8.1°±0.2° 2-theta. In another embodiment is provided a crystalline form of Compound 1, wherein the crystalline form exhibits a further peak in an x-ray powder diffraction (XRPD) pattern at 9.6°±0.2° 2-theta. A further embodiment provides a crystalline form of Compound 1, wherein the crystalline form exhibits further peaks in an x-ray powder diffraction (XRPD) pattern at 5.7° 0.2° 2-theta, 19.7° ° 0.2° 2-theta, and 22.0° ° 0.2° 2-theta. In still a further embodiment is provided a crystalline form of Compound 1, wherein the crystalline form exhibits further peaks in an x-ray powder diffraction (XRPD) pattern at 9.8° 0.2° 2-theta, 15.2° ° 0.2° 2-theta, and 17.7° 0.2° 2-theta. In yet another embodiment is provided a crystalline form of Compound 1, wherein the crystalline form comprises a peak in a differential scanning calorimetry pattern of about 172° C. In yet another embodiment is provided a crystalline form of Compound 1, wherein the crystalline form comprises a peak in a differential scanning calorimetry pattern of from about 205° C. to about 210° C. In yet another embodiment is provided a crystalline form of Compound 1, wherein the crystalline form comprises a peak in a differential scanning calorimetry pattern of from about 206° C. to about 210° C., or from about 207° C. to about 210° C., or from about 208° C. to about 210° C., or from about 209° C. to about 210° C. In a further embodiment is provided a crystalline form of Compound 1, wherein the crystalline form exhibits a loss in mass in a thermal gravimetric analysis of less than about 1% upon heating the sample from about 25° C. to a temperature prior to melting. In a further embodiment is provided a crystalline form of Compound 1, wherein the crystalline form exhibits a loss in mass in a thermal gravimetric analysis of less than about 1% upon heating the sample from about 25° C. to about 380° C.

In another embodiment is provided a crystalline form of Compound 1, wherein the crystalline form exhibits a peak in an x-ray powder diffraction (XRPD) pattern at 7.7° 0.2° 2-theta. In yet another embodiment is provided a crystalline form of Compound 1, wherein the crystalline form exhibits further peaks in an x-ray powder diffraction (XRPD) pattern at 13.7° ° 0.2° 2-theta and 19.2° ° 0.2° 2-theta. In a still further embodiment is provided a crystalline form of Compound 1, wherein the crystalline form exhibits further peaks in an x-ray powder diffraction (XRPD) pattern at 5.5° 0.2° 2-theta, 8.6° ° 0.2° 2-theta, 15.9°±0.2° 2-theta, 19.9°±0.2° 2-theta, and 24.10° 0.2° 2-theta. Another embodiment provides a crystalline form of Compound 1, wherein the crystalline form exhibits further peaks in an x-ray powder diffraction (XRPD) pattern at 10.6° ° 0.2° 2-theta, 11.0° ° 0.2° 2-theta, 15.4° ° 0.2° 2-theta, 21.0° ° 0.2° 2-theta, and 26.3° ° 0.2° 2-theta. A further embodiment provides a crystalline form of Compound 1, wherein the crystalline form comprises a peak in a differential scanning calorimetry pattern of from about 203° C. to about 210° C. In yet another embodiment is provided a crystalline form of Compound 1, wherein the crystalline form comprises a peak in a differential scanning calorimetry pattern of from about 203° C. to about 208° C., or from about 203° C. to about 206° C., or from about 203° C. to about 205° C. In another embodiment is provided a crystalline form of Compound 1, wherein the crystalline form exhibits a loss in mass in a thermal gravimetric analysis of less than about 2% upon heating the sample from about 25° C. to about 380° C. In another embodiment is provided a crystalline form of Compound 1, wherein the crystalline form exhibits a loss in mass in a thermal gravimetric analysis of less than about 2% upon heating the sample from about 25° C. to about 210° C.

In another embodiment is provided a crystalline form of Compound 1, wherein the crystalline form exhibits a peak in an x-ray powder diffraction (XRPD) pattern at 7.7° 0.2° 2-theta. In yet another embodiment is provided a crystalline form of Compound 1, wherein the crystalline form exhibits further a peak in an x-ray powder diffraction (XRPD) pattern at 15.4°±0.2° 2-theta. In yet another embodiment is provided a crystalline form of Compound 1, wherein the crystalline form exhibits further a peak in an x-ray powder diffraction (XRPD) pattern at 19.2°±0.2° 2-theta. In yet another embodiment is provided a crystalline form of Compound 1, wherein the crystalline form exhibits a further peak in an x-ray powder diffraction (XRPD) pattern at 13.7°±0.2° 2-theta. In a still further embodiment is provided a crystalline form of Compound 1, wherein the crystalline form exhibits further peaks in an x-ray powder diffraction (XRPD) pattern at 5.5°±0.2° 2-theta, 8.6°±0.2° 2-theta, 15.9°±0.2° 2-theta, 19.9°±0.2° 2-theta, and 24.1°±0.2° 2-theta. Another embodiment provides a crystalline form of Compound 1, wherein the crystalline form exhibits further peaks in an x-ray powder diffraction (XRPD) pattern at 10.6°±0.2° 2-theta, 11.0°±0.2° 2-theta, 21.0°±0.2° 2-theta, and 26.3°±0.2° 2-theta. A further embodiment provides a crystalline form of Compound 1, wherein the crystalline form comprises a peak in a differential scanning calorimetry pattern of from about 203° C. to about 210° C. A further embodiment provides a crystalline form of Compound 1, wherein the crystalline form comprises a peak in a differential scanning calorimetry pattern of from about 206° C. to about 210° C. In yet another embodiment is provided a crystalline form of Compound 1, wherein the crystalline form comprises a peak in a differential scanning calorimetry pattern of from about 203° C. to about 208° C., or from about 203° C. to about 206° C., or from about 203° C. to about 205° C. In another embodiment is provided a crystalline form of Compound 1, wherein the crystalline form exhibits a loss in mass in a thermal gravimetric analysis of less than about 2% upon heating the sample from about 25° C. to about 380° C. In another embodiment is provided a crystalline form of Compound 1, wherein the crystalline form exhibits a loss in mass in a thermal gravimetric analysis of less than about 2% upon heating the sample from about 25° C. to about 210° C.

In another embodiment is provided a crystalline form of Compound 1, wherein the crystalline form exhibits peaks in an x-ray powder diffraction (XRPD) pattern at 7.7°±0.2° 2-theta and 15.4°±0.2° 2-theta. In another embodiment is provided a crystalline form of Compound 1, wherein the crystalline form further exhibits a peak in an x-ray powder diffraction (XRPD) pattern at 19.2°±0.2° 2-theta. In yet another embodiment is provided a crystalline form of Compound 1, wherein the crystalline form exhibits a further peak in an x-ray powder diffraction (XRPD) pattern at 13.7°±0.2° 2-theta. In a still further embodiment is provided a crystalline form of Compound 1, wherein the crystalline form exhibits further peaks in an x-ray powder diffraction (XRPD) pattern at 5.5°±0.2° 2-theta, 8.6°±0.2° 2-theta, 15.9°±0.2° 2-theta, 19.9°±0.2° 2-theta, and 24.1°±0.2° 2-theta. Another embodiment provides a crystalline form of Compound 1, wherein the crystalline form exhibits further peaks in an x-ray powder diffraction (XRPD) pattern at 10.6°±0.2° 2-theta, 11.0°±0.2° 2-theta, 21.0°±0.2° 2-theta, and 26.3°±0.2° 2-theta. A further embodiment provides a crystalline form of Compound 1, wherein the crystalline form comprises a peak in a differential scanning calorimetry pattern of from about 203° C. to about 210° C. A further embodiment provides a crystalline form of Compound 1, wherein the crystalline form comprises a peak in a differential scanning calorimetry pattern of from about 206° C. to about 210° C. In yet another embodiment is provided a crystalline form of Compound 1, wherein the crystalline form comprises a peak in a differential scanning calorimetry pattern of from about 203° C. to about 208° C., or from about 203° C. to about 206° C., or from about 203° C. to about 205° C. In another embodiment is provided a crystalline form of Compound 1, wherein the crystalline form exhibits a loss in mass in a thermal gravimetric analysis of less than about 2% upon heating the sample from about 25° C. to about 380° C. In another embodiment is provided a crystalline form of Compound 1, wherein the crystalline form exhibits a loss in mass in a thermal gravimetric analysis of less than about 2% upon heating the sample from about 25° C. to about 210° C.

In another embodiment is provided a crystalline form of Compound 1, wherein the crystalline form exhibits peaks in an x-ray powder diffraction (XRPD) pattern at 7.7°±0.2° 2-theta and 19.2°±0.2° 2-theta. In another embodiment is provided a crystalline form of Compound 1, wherein the crystalline form further exhibits a peak in an x-ray powder diffraction (XRPD) pattern at 15.4°±0.2° 2-theta. In yet another embodiment is provided a crystalline form of Compound 1, wherein the crystalline form exhibits a further peak in an x-ray powder diffraction (XRPD) pattern at 13.7°±0.2° 2-theta. In a still further embodiment is provided a crystalline form of Compound 1, wherein the crystalline form exhibits further peaks in an x-ray powder diffraction (XRPD) pattern at 5.5°±0.2° 2-theta, 8.6°±0.2° 2-theta, 15.9°±0.2° 2-theta, 19.9°±0.2° 2-theta, and 24.1°±0.2° 2-theta. Another embodiment provides a crystalline form of Compound 1, wherein the crystalline form exhibits further peaks in an x-ray powder diffraction (XRPD) pattern at 10.6°±0.2° 2-theta, 11.0°±0.2° 2-theta, 21.0°±0.2° 2-theta, and 26.3°±0.2° 2-theta. A further embodiment provides a crystalline form of Compound 1, wherein the crystalline form comprises a peak in a differential scanning calorimetry pattern of from about 203° C. to about 210° C. A further embodiment provides a crystalline form of Compound 1, wherein the crystalline form comprises a peak in a differential scanning calorimetry pattern of from about 206° C. to about 210° C. In yet another embodiment is provided a crystalline form of Compound 1, wherein the crystalline form comprises a peak in a differential scanning calorimetry pattern of from about 203° C. to about 208° C., or from about 203° C. to about 206° C., or from about 203° C. to about 205° C. In another embodiment is provided a crystalline form of Compound 1, wherein the crystalline form exhibits a loss in mass in a thermal gravimetric analysis of less than about 2% upon heating the sample from about 25° C. to about 380° C. In another embodiment is provided a crystalline form of Compound 1, wherein the crystalline form exhibits a loss in mass in a thermal gravimetric analysis of less than about 2% upon heating the sample from about 25° C. to about 210° C.

In further embodiments are provided crystalline forms of Compound 1 as disclosed herein, wherein the crystalline forms exhibit less than about 10% degradation when the crystalline forms are stored at 25° C. and 60% relative humidity for at least 7 days, at least 1 month, at least 3 months, and at least 6 months. Other embodiments provide crystalline forms of Compound 1 as disclosed herein, wherein the crystalline forms exhibit less than about 1%, or less than about 2%, or less than about 3%, or less than about 4%, or less than about 5%, or less than about 6%, or less than about 7%, or less than about 8%, or less than about 9% degradation when the crystalline form is stored at 25° C. and 60% relative humidity for at least 7 days, at least 1 month, at least 3 months, or at least 6 months.

In a further embodiment is provided a crystalline form of Compound 1, wherein the crystalline form (a) exhibits a peak in an x-ray powder diffraction (XRPD) pattern at 8.1°±0.2° 2-theta, and (b) exhibits less than about 10% degradation when the crystalline form are stored at 25° C. and 60% relative humidity for at least 7 days, at least 1 month, at least 3 months, or at least 6 months. In a further embodiment is provided a crystalline form of Compound 1, wherein the crystalline form (a) exhibits peaks in an x-ray powder diffraction (XRPD) pattern at 9.6°±0.2° 2-theta, 5.7°±0.2° 2-theta, 19.7°±0.2° 2-theta, and 22.0°±0.2° 2-theta, and (b) less than about 10% degradation when the crystalline form are stored at 25° C. and 60% relative humidity for at least 7 days, at least 1 month, at least 3 months, or at least 6 months. Other embodiments provide such crystalline forms of Compound 1, wherein the crystalline forms exhibit less than about 1%, or less than about 2%, or less than about 3%, or less than about 4%, or less than about 5%, or less than about 6%, or less than about 7%, or less than about 8%, or less than about 9% degradation when the crystalline form is stored at 25° C. and 60% relative humidity for at least 7 days, at least 1 month, at least 3 months, or at least 6 months.

In a further embodiment is provided a crystalline form of Compound 1, wherein the crystalline form (a) exhibits a peak in an x-ray powder diffraction (XRPD) pattern at 7.7°±0.2° 2-theta, and (b) exhibits less than about 10% degradation when the crystalline form are stored at 25° C. and 60% relative humidity for at least 7 days, at least 1 month, at least 3 months, or at least 6 months. In a further embodiment is provided a crystalline form of Compound 1, wherein the crystalline form (a) exhibits peaks in an x-ray powder diffraction (XRPD) pattern at 7.7°±0.2° 2-theta, 13.7°±0.2° 2-theta, and 19.2°±0.2° 2-theta, and (b) less than about 10% degradation when the crystalline form are stored at 25° C. and 60% relative humidity for at least 7 days, at least 1 month, at least 3 months, or at least 6 months. Other embodiments provide such crystalline forms of Compound 1, wherein the crystalline forms exhibit less than about 1%, or less than about 2%, or less than about 3%, or less than about 4%, or less than about 5%, or less than about 6%, or less than about 7%, or less than about 8%, or less than about 9% degradation when the crystalline form is stored at 25° C. and 60% relative humidity for at least 7 days, at least 1 month, at least 3 months, or at least 6 months.

Further embodiments provide crystalline forms of Compound 1 as disclosed herein, wherein the crystalline forms exhibit less than about 10% degradation when the crystalline forms are stored at 40° C. and 75% relative humidity for at least 7 days, at least 1 month, at least 3 months, or at least 6 months. Further embodiments provide crystalline forms of Compound 1 as disclosed herein, wherein the crystalline forms exhibit less than about 1%, or less than about 2%, or less than about 3%, or less than about 4%, or less than about 5%, or less than about 6%, or less than about 7%, or less than about 8%, or less than about 9% degradation when the crystalline form is stored at 40° C. and 75% relative humidity for at least 7 days, at least 1 month, at least 3 months, or at least 6 months.

In a further embodiment is provided a crystalline form of Compound 1, wherein the crystalline form (a) exhibits a peak in an x-ray powder diffraction (XRPD) pattern at 8.1°±0.2° 2-theta, and (b) exhibits less than about 10% degradation when the crystalline form are stored at 40° C. and 75% relative humidity for at least 7 days, at least 1 month, at least 3 months, or at least 6 months. In a further embodiment is provided a crystalline form of Compound 1 as disclosed herein, wherein the crystalline form (a) exhibits peaks in an x-ray powder diffraction (XRPD) pattern at 9.6°±0.2° 2-theta, 5.7°±0.2° 2-theta, 19.7°±0.2° 2-theta, and 22.0°±0.2° 2-theta, and (b) less than about 10% degradation when the crystalline form are stored at 40° C. and 75% relative humidity for at least 7 days, at least 1 month, at least 3 months, or at least 6 months. Other embodiments provide such crystalline forms of Compound 1, wherein the crystalline forms exhibit less than about 1%, or less than about 2%, or less than about 3%, or less than about 4%, or less than about 5%, or less than about 6%, or less than about 7%, or less than about 8%, or less than about 9% degradation when the crystalline form is stored at 40° C. and 75% relative humidity for at least 7 days, at least 1 month, at least 3 months, or at least 6 months.

In a further embodiment is provided a crystalline form of Compound 1, wherein the crystalline form (a) exhibits a peak in an x-ray powder diffraction (XRPD) pattern at 7.7°±0.2° 2-theta, and (b) exhibits less than about 10% degradation when the crystalline form are stored at 40° C. and 75% relative humidity for at least 7 days, at least 1 month, at least 3 months, or at least 6 months. In a further embodiment is provided a crystalline form of Compound 1, wherein the crystalline form (a) exhibits peaks in an x-ray powder diffraction (XRPD) pattern at 7.7°±0.2° 2-theta, 13.7°±0.2° 2-theta, and 19.2°±0.2° 2-theta, and (b) less than about 10% degradation when the crystalline form are stored at 40° C. and 75% relative humidity for at least 7 days, at least 1 month, at least 3 months, or at least 6 months. Other embodiments provide such crystalline forms of Compound 1, wherein the crystalline forms exhibit less than about 1%, or less than about 2%, or less than about 3%, or less than about 4%, or less than about 5%, or less than about 6%, or less than about 7%, or less than about 8%, or less than about 9% degradation when the crystalline form is stored at 40° C. and 75% relative humidity for at least 7 days, at least 1 month, at least 3 months, or at least 6 months.

Other embodiments provide crystalline forms of Compound 1 as disclosed herein, wherein the crystalline forms exhibit less than about 10% degradation when the crystalline forms are stored at 60° C. for at least one week. Further embodiments provide crystalline forms of Compound 1, wherein the crystalline form exhibits less than about 1%, or less than about 2%, or less than about 3%, or less than about 4%, or less than about 5%, or less than about 6%, or less than about 7%, or less than about 8%, or less than about 9% degradation when the crystalline forms are stored at 60° C. for at least one week.

Further provided herein are pharmaceutical compositions comprising an amount of any of the crystalline forms of Compound 1 disclosed herein, and at least one pharmaceutically acceptable excipient.

In another embodiment is provided a method of inhibiting PRC2 activity in a subject in need thereof, comprising administering to the subject an effective amount of any of the crystalline forms of Compound 1 disclosed herein. Further provided herein are methods of inhibiting PRC2 activity in a subject in need thereof, comprising administering to the subject an effective amount of a pharmaceutical composition disclosed herein.

In yet another embodiment is provided a crystalline form of Compound 1 as disclosed herein for use in a method for inhibiting PRC2 activity in a subject in need thereof. In some uses, the PRC2 activity in the subject in need thereof is associated with the subject having a PRC2-associated cancer.

In further embodiments are provided methods of treating cancer in a subject, comprising administering to the subject a therapeutically effective amount of any of the crystalline forms of Compound 1 disclosed herein. In some embodiments are provided such methods of treating cancer, wherein the cancer is selected from the group consisting of Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochondroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma. In some embodiments, the cancer is a PRC2-associated cancer. In further embodiments, the cancer is prostate cancer, breast cancer, skin cancer, bladder cancer, liver cancer, pancreatic cancer, or head and neck cancer. In an embodiment, the cancer is prostate cancer. In an embodiment, the prostate cancer is metastatic prostate cancer, metastatic castration-resistant prostate cancer, localized high risk prostate cancer, recurrent prostate cancer, non-metastatic CRPC (nmCRPC), non-metastatic castration-sensitive prostate cancer, or metastatic castration-sensitive prostate cancer. In some embodiments, the cancer is breast cancer. In some embodiments, the cancer is skin cancer. In some embodiments, the cancer is bladder cancer. In some embodiments, the cancer is liver cancer. In some embodiments, the cancer is pancreatic cancer. In some embodiments, the cancer is cancer is head and neck cancer.

In yet another embodiment is provided a crystalline form of Compound 1 as disclosed herein for use in a method for treating cancer in a subject in need thereof. In some uses, the cancer in the subject in need thereof is a PRC2-associated cancer. In further embodiments, the cancer is prostate cancer, breast cancer, skin cancer, bladder cancer, liver cancer, pancreatic cancer, or head and neck cancer. In an embodiment, the cancer is prostate cancer. In an embodiment, the prostate cancer is metastatic prostate cancer, metastatic castration-resistant prostate cancer, localized high risk prostate cancer, recurrent prostate cancer, non-metastatic CRPC (nmCRPC), non-metastatic castration-sensitive prostate cancer, or metastatic castration-sensitive prostate cancer. In some embodiments, the cancer is breast cancer. In some embodiments, the cancer is skin cancer. In some embodiments, the cancer is bladder cancer. In some embodiments, the cancer is liver cancer. In some embodiments, the cancer is pancreatic cancer. In some embodiments, the cancer is cancer is head and neck cancer.

Also provided herein are pharmaceutical compositions comprising an amount of a crystalline form of Compound 1 as disclosed herein, and one or more pharmaceutically acceptable excipients. Further provided herein are such pharmaceutical compositions, wherein the one or more pharmaceutically acceptable excipients comprises one or more diluents, binders, disintegrants, lubricants, antiadhesives, glidants, coloring agents, flavors, sweeteners, coating agents, plasticizers wetting agents, buffers, or adsorbents.

Also provided herein are pharmaceutical compositions comprising an amount of a crystalline form of Compound 1 as disclosed herein, and one or more pharmaceutically acceptable excipients, for use in a method for treating cancer in a subject in need thereof. In some uses, the cancer in the subject in need thereof is a PRC2-associated cancer. In further embodiments, the cancer is prostate cancer, breast cancer, skin cancer, bladder cancer, liver cancer, pancreatic cancer, or head and neck cancer. In an embodiment, the cancer is prostate cancer. In an embodiment, the prostate cancer is metastatic prostate cancer, metastatic castration-resistant prostate cancer, localized high risk prostate cancer, recurrent prostate cancer, non-metastatic CRPC (nmCRPC), non-metastatic castration-sensitive prostate cancer, or metastatic castration-sensitive prostate cancer. In some embodiments, the cancer is breast cancer. In some embodiments, the cancer is skin cancer. In some embodiments, the cancer is bladder cancer. In some embodiments, the cancer is liver cancer. In some embodiments, the cancer is pancreatic cancer. In some embodiments, the cancer is cancer is head and neck cancer.

Among the one or more diluents that may be used are lactose, mannitol, xylitol, microcrystalline cellulose, dibasic calcium phosphate and starch. In some embodiments, the one or more diluents comprises from about 1% to about 80%, or from about 10% to about 80%, or from about 10% to about 70%, or from about 15% to about 80%, or from about 20% to about 80%, or from about 15% to about 75%, or from about 20% to about 75%, or from about 25% to about 75%, or from about 50% to about 80%, or from about 50% to about 75%, or from about 60% to about 80%, or from about 60% to 75% of the total weight of the pharmaceutical compositions. In some embodiments, the diluent is lactose. In some embodiments, the diluent is mannitol. In some embodiments, the diluent is xylitol. In some embodiments, the diluent is microcrystalline cellulose. In some embodiments, the diluent is dibasic calcium phosphate. In some embodiments, the diluent is starch. In some embodiments, the diluent is isomalt. In some embodiments, the diluent is silicified microcrystalline cellulose. In some embodiments, the diluent is pregelatinized starch.

Also provided herein are such pharmaceutical compositions, wherein the one or more pharmaceutically acceptable excipients comprises one or more binders, wherein the one or more binders comprises from about 1% to about 80%, or from about 10% to about 80%, or from about 10% to about 70%, or from about 15% to about 80%, or from about 20% to about 80%, or from about 15% to about 75%, or from about 20% to about 75%, or from about 25% to about 75%, or from about 50% to about 80%, or from about 50% to about 75%, or from about 60% to about 80%, or from about 60% to about 75% of the total weight of the pharmaceutical compositions. In some embodiments, the one or more binders is selected from selected from methyl cellulose, microcrystalline cellulose, starch, and gums such as guar gum, and tragacanth, or a mixture thereof.

Also provided herein are such pharmaceutical compositions, wherein the one or more pharmaceutically acceptable excipients comprises one or more disintegrants, and wherein the one or more disintegrants comprises from about 0.1% to about 10%, or from about 0.1% to about 5%, or from about 0.1% to about 4%, or from about 0.1% to about 3%, or from about 0.1% to about 2%, or from about 0.1% to about 1%, or from about 0.1% to about 0.75%, or from about 0.2% to about 1%, or from about 0.3% to about 1%, or from about 0.4% to about 1%, or from about 0.2% to about 0.8%, or from about 0.3% to about 0.75%, or from about 0.3% to about 0.7%, or from about 0.3% to about 0.6% by weight of the total weight of the pharmaceutical compositions. In some embodiments the one or more disintegrants is selected from starch, sodium starch glycolate, sodium alginate, carboxymethylcellulose sodium, methyl cellulose, croscarmellose sodium and crospovidone, or mixtures thereof. In some embodiments, the disintegrant is starch. In some embodiments, the disintegrant is sodium starch glycolate. In some embodiments, the disintegrant is sodium alginate. In some embodiments, the disintegrant is carboxymethylcellulose sodium. In some embodiments, the disintegrant is methyl cellulose. In some embodiments, the disintegrant is croscarmellose sodium. In some embodiments, the disintegrant is crospovidone. In some embodiments, the disintegrant is methylcellulose. In some embodiments, the disintegrant is carboxymethylcellulose sodium.

Also provided herein are such pharmaceutical compositions, wherein the one or more pharmaceutically acceptable excipients comprises one or more lubricants, and wherein the one or more lubricants comprises from about 0.1% to about 10%, or from about 0.1% to about 5%, or from about 0.1% to about 4%, or from about 0.1% to about 3%, or from about 0.1% to about 2%, or from about 0.10% to about 10%, or from about 0.1% to about 0.75%, or from about 0.2% to about 10%, or from about 0.3% to about 1%, or from about 0.4% to about 1%, or from about 0.2% to about 0.8%, or from about 0.3% to about 0.75%, or from about 0.3% to about 0.7%, or from about 0.3% to about 0.6% by weight of the total weight of the pharmaceutical compositions. In a further embodiment, the one or more lubricants is selected from magnesium stearate, calcium stearate, sodium stearyl fumarate, and stearic acid, or mixtures thereof. In some embodiments, the lubricant is magnesium stearate. In some embodiments, the lubricant is calcium stearate. In some embodiments, the lubricant is sodium stearyl fumarate. In some embodiments, the lubricant is and stearic acid. In some embodiments, the lubricant is glyceryl behenate. In some embodiments, the lubricant is glycerin monostearate. In some embodiments, the lubricant is magnesium lauryl sulfate.

In some embodiments, the pharmaceutical compositions disclosed herein may comprise additional excipients including, but not limited, to buffers or buffering agents, glidants, preservatives, and coloring agents. Additional excipients such as bulking agents, tonicity agents, and chelating agents are also within the scope of the embodiments.

Non-limiting examples of buffering agents include, but are not limited to, sodium bicarbonate, potassium bicarbonate, magnesium hydroxide, magnesium lactate, magnesium gluconate, aluminum hydroxide, aluminum hydroxide/sodium bicarbonate co precipitate, a mixture of an amino acid and a buffer, a mixture of aluminum glycinate and a buffer, a mixture of an acid salt of an amino acid and a buffer, and a mixture of an alkali salt of an amino acid and a buffer. Additional buffering agents include sodium citrate, sodium tartrate, sodium acetate, sodium carbonate, sodium polyphosphate, potassium polyphosphate, sodium pyrophosphate, potassium pyrophosphate, disodium hydrogenphosphate, dipotassium hydrogenphosphate, trisodium phosphate, tripotassium phosphate, sodium acetate, potassium metaphosphate, magnesium oxide, magnesium hydroxide, magnesium carbonate, magnesium silicate, calcium acetate, calcium glycerophosphate, calcium chloride, calcium hydroxide, calcium lactate, calcium carbonate, calcium bicarbonate, and other calcium salts.

In some embodiments, the pharmaceutical compositions disclosed herein may comprise a glidant. Suitable glidants include, but are not limited to, calcium phosphate tribasic, calcium silicate, cellulose, colloidal silicon dioxide, magnesium silicate, magnesium trisilicate, silicon dioxide, starch, talc, and the like. In some embodiments, the glidant is calcium phosphate tribasic. In some embodiments, the glidant is calcium silicate. In some embodiments, the glidant is cellulose. In some embodiments, the glidant is colloidal silicon dioxide. In some embodiments, the glidant is magnesium silicate. In some embodiments, the glidant is magnesium trisilicate. In some embodiments, the glidant is silicon dioxide. In some embodiments, the glidant is starch. In some embodiments, the glidant is talc.

In some embodiments, the pharmaceutical compositions disclosed herein may comprise a preservative. Preservatives include anti-microbials, anti-oxidants, and agents that enhance sterility. Exemplary preservatives include ascorbic acid, ascorbyl palmitate, BHA, BHT, citric acid, erythorbic acid, fumaric acid, malic acid, propyl gallate, sodium ascorbate, sodium bisulfate, sodium metabisulfite, sodium sulfite, parabens (methyl-, ethyl-, butyl-), benzoic acid, potassium sorbate, vanillin, and the like.

In some embodiments, the pharmaceutical compositions disclosed herein may comprise a coloring agent for identity and/or aesthetic purposes of the resultant liquid form. Suitable coloring agents illustratively include FD&C Red No. 3, FD&C Red No. 20, FD&C Red No. 40, FD&C Yellow No. 6, FD&C Blue No. 2, D&C Green No. 5, D&C Orange No. 5, caramel, ferric oxide, and mixtures thereof.

Additional excipients are contemplated in the pharmaceutical compositions disclosed herein. These additional excipients are selected based on function and compatibility with the pharmaceutical compositions described herein and may be found, for example in Remington: The Science and Practice of Pharmacy, Nineteenth Ed (Easton, PA: Mack Publishing Company, 1995); Hoover, John E., Remington's Pharmaceutical Sciences, (Easton, PA: Mack Publishing Co 1975); Liberman, H. A. and Lachman, L., Eds., Pharmaceutical Dosage Forms (New York, NY: Marcel Decker 1980); and Pharmaceutical Dosage Forms and Drug Delivery Systems, Seventh Ed (Lippincott Williams & Wilkins 1999), herein incorporated by reference in their entirety.

The pharmaceutical compositions disclosed herein may be in a form suitable for oral dosage to a subject in need. Suitable oral dosage forms include, for example, tablets, pills, sachets, or capsules of hard or soft gelatin, methylcellulose or of another suitable material easily dissolved in the digestive tract. Oral administration of a solid dose form may be, for example, presented in discrete units, such as hard or soft capsules, pills, cachets, lozenges, or tablets, each containing a predetermined amount of a crystalline form of Compound 1 as disclosed herein and one or more pharmaceutically acceptable excipients. In another embodiment, the oral administration may be in a powder or granule form. In another embodiment, the oral dose form is sub-lingual, such as, for example, a lozenge. Capsules or tablets may contain a controlled-release formulation. In the case of capsules, tablets, and pills, the dosage forms also may comprise buffering agents or may be prepared with enteric coatings. In another embodiment, oral administration may be in a liquid dose form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as wetting, emulsifying, suspending, flavoring (e.g., sweetening), and/or perfuming agents.

In another embodiment, the pharmaceutical compositions disclosed herein may comprise a parenteral dose form. “Parenteral administration” includes, for example, subcutaneous injections, intravenous injections, intraperitoneal injections, intramuscular injections, intrasternal injections, and infusion. Injectable preparations (e.g., sterile injectable aqueous or oleaginous suspensions) may be formulated according to the known art using suitable dispersing, wetting agents, and/or suspending agents.

In another embodiment, the pharmaceutical compositions disclosed herein may comprise a topical dose form. “Topical administration” includes, for example, transdermal administration, such as via transdermal patches or iontophoresis devices, intraocular administration, or intranasal or inhalation administration. Compositions for topical administration also include, for example, topical gels, sprays, ointments, and creams. A topical formulation may include a compound that enhances absorption or penetration of the active ingredient through the skin or other affected areas. When the compositions disclosed herein are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated; see, for example, J. Pharm. Sci., 88(10), 955-958, by Finnin and Morgan (October 1999).

For intranasal administration or administration by inhalation, the pharmaceutical compositions disclosed herein are conveniently delivered in the form of a solution or suspension from a pump spray container that is squeezed or pumped by the subject or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

Other carrier materials and modes of administration known in the pharmaceutical art may also be used. Pharmaceutical compositions disclosed herein may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pa., 1975; Liberman et al., Eds., Pharmaceutical Dosage Forms, Marcel Decker, New York, N.Y., 1980; and Kibbe et al., Eds., Handbook of Pharmaceutical Excipients (3.sup.rd Ed.), American Pharmaceutical Association, Washington, 1999.

The dose of the composition comprising a crystalline form of Compound 1 as described herein may differ, depending upon the subject's (e.g., human) condition, that is, stage of the disease, general health status, age, and other factors. Pharmaceutical compositions are administered in a manner appropriate to the disease to be treated (or prevented). An appropriate dose and a suitable duration and frequency of administration will be determined by such factors as the condition of the subject, the type and severity of the subject's disease, the particular form of the active ingredient, and the method of administration. In general, an appropriate dose and treatment regimen provides the composition(s) in an amount sufficient to provide therapeutic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the subject. Oral doses typically range from about 1.0 mg to about 1000 mg, one to four times, or more, per day.

Compound 1 may be prepared by methods known to those having ordinary skill in the art, including, but not limited to, the methods set forth in U.S. Pat. No. 11,091,495, the contents of which are hereby incorporated by reference for that purpose.

### EXAMPLES

### Example 1: Preparation of Compound 1

Compound 1 may be prepared according to the scheme below. Compounds (5-fluoro-2,3-dihydrobenzofuran-4-yl)methanamine (compound A), 8-bromo-5-chloroimidazo[1,2-c]pyrimidine-2-carbonitrile (Compound B), and N,N-dimethyl-1-(3-methyl-4-(4,4,5,5-tetramethyl-1,3,2-dioxaborolan-2-yl)phenyl)methanamine (Compound D) may be prepared by methods known to those having ordinary skill in the art, including, but not limited to, the methods set forth in U.S. Pat. No. 11,091,495, the contents of which are hereby incorporated by reference for that purpose.

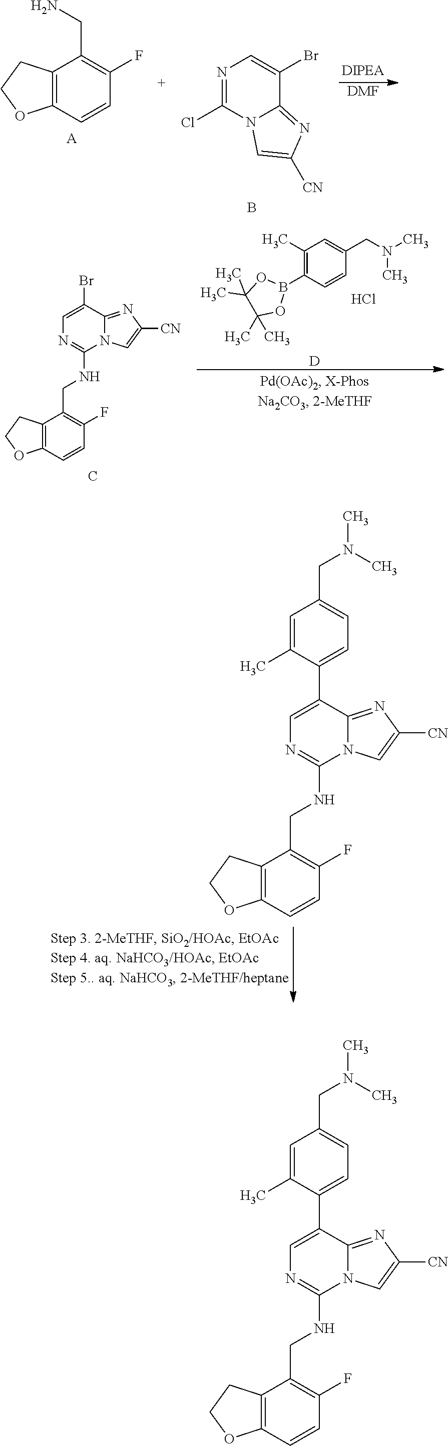

Step 1: A mixture of 5-fluoro-2,3-dihydrobenzofuran-4-yl)methanamine (compound A) and 8-bromo-5-chloroimidazo[1,2-c]pyrimidine-2-carbonitrile (Compound B) in N,N-dimethylformamide (DMF) was treated with diisopropylethylamine (DIPEA) and the mixture was stirred until the reaction was deemed complete. The resulting mixture was treated with ethanol and water, and then filtered to give 8-bromo-5-(((5-fluoro-2,3-dihydrobenzofuran-4-yl)methyl)amino)imidazo[1,2-c]pyrimidine-2-carbonitrile (Compound C).

Step 2: A mixture of 8-bromo-5-(((5-fluoro-2,3-dihydrobenzofuran-4-yl)methyl)amino)imidazo[1,2-c]pyrimidine-2-carbonitrile (Compound C), N,N-dimethyl-1-(3-methyl-4-(4,4,5,5-tetramethyl-1,3,2-dioxaborolan-2-yl)phenyl)methanamine hydrochloride (Compound D), palladium acetate (Pd(OAc)₂), 2-(dicyclohexylphosphino)-1′,2′,3′-triisopropyl-1,1′-biphenyl (X-Phos), aqueous sodium carbonate (aqueous Na₂CO₃), and 2-methyl tetrahydrofuran (2-MeTHF) was heated until the reaction was deemed complete. The mixture was then subjected to an aqueous workup into 2-MeTHF and the crude product was isolated by crystallization and filtration from 2-MeTHF/heptane to afford crude 8-(4-((dimethylamino)methyl)-2-methylphenyl)-5-(((5-fluoro-2,3-dihydrobenzofuran-4-yl)methyl)amino)imidazo[1,2-c]pyrimidine-2-carbonitrile (Compound 1).

Step 3: The crude 8-(4-((dimethylamino)methyl)-2-methylphenyl)-5-(((5-fluoro-2,3-dihydrobenzofuran-4-yl)methyl)amino)imidazo[1,2-c]pyrimidine-2-carbonitrile (Compound 1) was taken up in 2-MeTHF, passed through a pad of silica gel (SiO2), and then the solvent was switched to ethyl acetate (EtOAc). The resulting mixture was then treated with acetic acid (HOAc) and filtered to give the acetic acid salt of 8-(4-((dimethylamino)methyl)-2-methylphenyl)-5-(((5-fluoro-2,3-dihydrobenzofuran-4-yl)methyl)amino)imidazo[1,2-c]pyrimidine-2-carbonitrile.

Step 4: The acetic acid salt of 8-(4-((dimethylamino)methyl)-2-methylphenyl)-5-(((5-fluoro-2,3-dihydrobenzofuran-4-yl)methyl)amino)imidazo[1,2-c]pyrimidine-2-carbonitrile from Step 3 was converted into the free base by treatment with aqueous NaHCO₃ in ethyl acetate. The resulting solution of 8-(4-((dimethylamino)methyl)-2-methylphenyl)-5-(((5-fluoro-2,3-dihydrobenzofuran-4-yl)methyl)amino)imidazo[1,2-c]pyrimidine-2-carbonitrile in EtOAc was then treated with HOAc and the resulting mixture was filtered to give the acetic acid salt of 8-(4-((dimethylamino)methyl)-2-methylphenyl)-5-(((5-fluoro-2,3-dihydrobenzofuran-4-yl)methyl)amino)imidazo[1,2-c]pyrimidine-2-carbonitrile.

Step 5: The acetic acid salt of 8-(4-((dimethylamino)methyl)-2-methylphenyl)-5-(((5-fluoro-2,3-dihydrobenzofuran-4-yl)methyl)amino)imidazo[1,2-c]pyrimidine-2-carbonitrile from Step 4 was converted into the free base by treatment with aqueous NaHCO₃ in 2-MeTHF, the resulting solution was treated with heptane, and the resulting mixture was filtered to give 8-(4-((dimethylamino)methyl)-2-methylphenyl)-5-(((5-fluoro-2,3-dihydrobenzofuran-4-yl)methyl)amino)imidazo[1,2-c]pyrimidine-2-carbonitrile (Compound 1).

### Example 2A: Preparation of Crystalline Form 1 of Compound 1

150 μL of methanol was added to 50 mg of the free base of Compound 1 and the resulting slurry was stirred at room temperature for one day. The resulting solids were vacuum filtered and dried under ambient condition overnight to afford Form 1 of Compound 1.

### Example 2B: Preparation of Crystalline Form 2 of Compound 1

400 mg of the free base of Compound 1 was dissolved in 1.5 mL of 2-methyltetrahydrofuran at 50° C., to which was added 1.5 mL n-heptane at about 47° C., and the resulting mixture was cooled 10° C. The resulting solids were vacuum filtered and allowed to air dry overnight under ambient conditions to afford Form 2 of Compound 1.

### Example 2C: Preparation of Crystalline Form 2 of Compound 1

A quantity of the free base of Compound 1 was dissolved in 2-methyltetrahydrofuran (10 volumes) and then distilled to 3 volumes. The temperature of the solution was adjusted to about 25° C. and the resulting slurry was stirred for greater than 30 minutes. To the slurry was added n-heptane (7 volumes) over 2 hours and the resulting mixture was stirred for greater than 4 hours. The resulting solid was filtered, the filter cake was washed with 30% 2-methyltetrahydrofuran/heptane (2 volumes) and dried in a vacuum oven to provide Form 2 of Compound 1.

### Example 2D: Preparation of Crystalline Form 2 of Compound 1

A reactor was evacuated and charged with nitrogen to atmospheric pressure. The reactor was then charged with a solution of Compound 1 (approximately 2.41 kg as determined by solution assay using HPLC) in 2-methyltetrahydrofuran (2-MeTHF, 36 kg, 15 volumes) and the batch was concentrated to a batch volume of about 5 L (about 2 volumes) via distillation under reduced pressure. The resulting solution was adjusted to about 25° C. and then n-heptane (0.4 kg, 0.2 volumes) was added in portions over a period of about 3 hours. The resulting solution was then seeded with Compound 1 Form 2 (9 g, 0.4 wt %), the resulting mixture was stirred for about 1.3 hours, and then additional n-heptane (24 kg, 10 volumes) was added over about 6 hours. The resulting slurry was stirred for about 4.25 hours at 25° C. and then filtered. The reactor was then rinsed with n-heptane (5.8 kg, 2.5 V), and this mixture was rinsed forward to the filter cake, which was deliquored and the solids were dried under reduced pressure at 40° C. and 50 SC for 19 hours to provide 2.48 kg of Form 2 of Compound 1.

### Example 3: X-Ray Powder Diffraction (XRPD) Analysis of Form 1 and Form 2 of Compound 1

XRPD analyses of crystalline polymorphic forms of Compound 1 were performed using Panalytical X'pert³ X-ray powder diffractometer. Samples were spread on the middle of a zero-background Si holder. The 2-theta position was calibrated against a Panalytical Si reference standard disc. The parameters used for the analyses are set forth in Table 1.

TABLE 1

| Parameter | Reflection Mode |
|---|---|
| X-ray wavelength | Cu, kα |
| | Kα1 (angstroms): 1.540598; Kα2 (angstroms): |
| | 1.544426 |
| | Kα2/Kα1 intensity ratio: 0.50 |
| X-ray tube setting | 45 kV, 40 mA |
| Divergence slit | ⅛° |
| Scan mode | Continuous |
| Scan range (°2-theta) | 3° to 40° |
| Scan step time | 18.87 |
| (seconds) | |
| Step size (°2-theta) | 0.0131 |
| Test time | About 4 minutes, 15 seconds |

Polymorphic Form 1 of Compound 1 was analyzed by XRPD as set forth above and exhibited the peaks set forth in Table 2. The error associated with each °2-theta position was determined to be ±0.2° theta.

TABLE 2

| Position (°2-theta) | Relative intensity (%) |
|---|---|
| 5.7 | 23.83 |
| 8.1 | 100 |
| 9.6 | 32.45 |
| 9.8 | 17.88 |
| 12.9 | 18.43 |
| 15.2 | 18.62 |
| 17.7 | 18.68 |
| 19.7 | 22.92 |
| 22.0 | 22.15 |

Polymorphic Form 2 of Compound 1 was analyzed by XRPD as set forth above and exhibited the peaks set forth in Table 3. The error associated with each °2-theta position was determined to be ±0.2°-theta.

TABLE 3

| Position (°2-theta) | Relative intensity (%) |
|---|---|
| 5.5 | 70.17 |
| 7.7 | 100 |
| 8.6 | 62.56 |
| 10.6 | 55.03 |
| 11.0 | 41.46 |
| 13.7 | 85.08 |
| 15.4 | 47.42 |
| 15.9 | 60.49 |
| 19.2 | 78.68 |
| 19.9 | 60.93 |
| 21.0 | 50.16 |
| 24.1 | 68.95 |
| 25.3 | 38.36 |
| 26.3 | 57.53 |

### Example 4: Thermal Gravimetric Analyses and Differential Scanning Calorimetry Analyses of Form 1 and Form 2 of Compound 1

Thermal gravimetric analysis (TGA) data were collected using TA Discovery TGA 550 TGA from TA Instruments, and differential scanning calorimetry (DSC) analyses were performed using a TA Q2000 DSC from TA Instruments using the parameters set forth in Table 4.

TABLE 4

| Parameter | TGA | DSC |
|---|---|---|
| Method | Ramp | Ramp |
| Sample pan | Platinum, open | Aluminum, crimped |
| Temperature range | Room temperature to | Room temperature to |
| | about 380° C. | about 380° C. |
| Heating rate | 10° C./minute | 10° C./minute |
| Purge gas | Nitrogen | Nitrogen |

A thermal gravimetric analysis (TGA) of a sample of Form 1 of Compound 1, when conducted under the conditions set forth in Table 4, exhibited a weight loss of about 1% upon heating the sample from room temperature to about the onset of melting (about 207° C.). A differential scanning calorimetry (DSC) analysis of Form 1 of Compound 1, when conducted under the conditions set forth in Table 4, exhibited peaks at between about 170° C. and 172° C., and between about 207° C. and 208° C.

A thermal gravimetric analysis (TGA) of a sample of Form 2 of Compound 1, when conducted under the conditions set forth in Table 4, exhibited a weight loss of about 2% upon heating the sample from room temperature to about the onset of melting (about 204° C.). A differential scanning calorimetry (DSC) analysis of Form 2 of Compound 1, when conducted under the conditions set forth in Table 4, exhibited a peak between about 203° C. and 204° C.

### Example 5: Stability of Form 2 of Compound 1

To measure stability of the Form 2 of Compound 1 under storage conditions, samples of Form 2 of Compound 1 were placed in double low-density polyethylene bags with desiccant between the bags and each was placed into a high-density polyethylene drum. One drum was stored at a temperature of 25° C. and 60% relative humidity (RH), and a second drum was stored at a temperature of 40° C. and 75% RH. Samples of the material were taken from both drums at 1 month, 3 months, and 6 months and the samples were analyzed for the presence of impurities. The amount of the Form 2 of Compound 1, and the amount of any impurities, in each sample was measured by ultra-performance liquid chromatography (UPLC) using the test conditions set and solvent gradients forth in Table 5 and Table 6. The samples were tested to determine the amount remaining of Form 2 at each time point, and the measurements were conducted by x-ray powder diffraction (XRPD) according to USP <941>.

TABLE 5

| Column | Agilent BONUS RP RRHD, 2.1 × 150 | |
|---|---|---|
| | mm, 1.8 μm | |
| Flow (mL/min) | 0.4 | mL/min |
| Run time (min) | 60 | min |
| Injector volume (μL) | 1 | μL |
| Column temperature (° C.) | 40° | C. |
| Detection wavelength | 220 | nm |

| | |
|---|---|
| Compound 1 retention time | 20.0 |
| (min) | |
| Needle wash | Acetonitrile |
| Mobile Phase A | 0.1% trifluoroacetic acid (TFA) in water |
| | (v/v) |
| Mobile Phase B | 0.1% TFA in acetonitrile (v/v) |
| Diluent | Acetonitrile:water (7:3, v/v) |

TABLE 6

| Time (min) | Mobile Phase A % | Mobile Phase B % |
|---|---|---|
| Initial | 98 | 2 |
| 1.00 | 98 | 2 |
| 30.00 | 45 | 55 |
| 45.00 | 5 | 95 |
| 50.00 | 5 | 95 |
| 50.10 | 98 | 2 |
| 60.00 | 98 | 2 |

The results of the stability tests for Form 2 of Compound 1 under both storage conditions are set forth in Table 7.

TABLE 7

| | | | | |
|---|---|---|---|---|
| | Initial (Time = | Time = 1 | Time = 3 | Time = 6 |
| Test Parameter | zero) | month | months | months |

Storage Conditions = 25° C. and 60% RH

| | | | | |
|---|---|---|---|---|
| Total impurities | 1.4 | 1.4 | 1.4 | 1.4 |
| present (% area | | | | |
| measured by | | | | |
| UPLC) | | | | |
| XRPD | Crystalline | Crystalline | Crystalline | Crystalline |
| | | (consistent with | (consistent with | (consistent with |
| | | T = 0) | T = 0) | T = 0) |

Storage Conditions = 40° C. and at 75% RH

| | | | | |
|---|---|---|---|---|
| Total impurities | 1.4 | 1.4 | 1.4 | 1.4 |
| present (% area | | | | |
| measured by | | | | |
| UPLC) | | | | |
| XRPD | Crystalline | Crystalline | Crystalline | Crystalline |
| | | (consistent with | (consistent with | (consistent with |
| | | T = 0) | T = 0) | T = 0) |

The results demonstrated that Form 2 of Compound 1 was stable up to 6 months when stored at 25° C. and 60% RH, and was stable up to 6 months when stored at 40° C. and at 75% RH.

### EMBODIMENTS

Embodiment 1: A crystalline form of Compound 1:

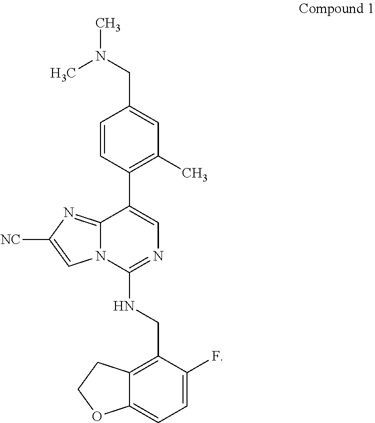 [SMILES: Cc1cc(CN(C)C)ccc1-c1cnc(NCc2c(F)ccc3c2CCO3)n2cc(C#N)nc12]

Embodiment 2: The crystalline form of Compound 1 according to embodiment 1, wherein the crystalline form is an anhydrous form.

Embodiment 3: The crystalline form of Compound 1 according to embodiment 1, wherein the crystalline form exhibits a peak in an x-ray powder diffraction (XRPD) pattern at 8.1°±0.2° 2-theta.

Embodiment 4: The crystalline form of Compound 1 according to embodiment 3, wherein the crystalline form exhibits a further peak in an x-ray powder diffraction (XRPD) pattern at 9.6°±0.2° 2-theta.

Embodiment 5: The crystalline form of Compound 1 according to embodiment 4, wherein the crystalline form exhibits further peaks in an x-ray powder diffraction (XRPD) pattern at 5.7°±0.2° 2-theta, 19.7°±0.2° 2-theta, and 22.0°±0.2° 2-theta.

Embodiment 6: The crystalline form of Compound 1 according to embodiment 5, wherein the crystalline form exhibits further peaks in an x-ray powder diffraction (XRPD) pattern at 9.8°±0.2° 2-theta, 15.2°±0.2° 2-theta, and 17.7°±0.2° 2-theta.

Embodiment 7: The crystalline form of Compound 1 according to any one of embodiments 1 to 6, wherein the crystalline form comprises a peak in a differential scanning calorimetry pattern of from about 205° C. to about 210° C.

Embodiment 8: The crystalline form of Compound 1 according to any one of embodiments 1 to 7, wherein the crystalline form exhibits a loss in mass in a thermal gravimetric analysis of less than about 1% upon heating the sample from about 25° C. to about 380° C.

Embodiment 9: The crystalline form of Compound 1 according to embodiment 1, wherein the crystalline form exhibits a peak in an x-ray powder diffraction (XRPD) pattern at 7.7°±0.2° 2-theta.

Embodiment 10: The crystalline form of Compound 1 according to embodiment 9, wherein the crystalline form exhibits further peaks in an x-ray powder diffraction (XRPD) pattern at 13.7°±0.2° 2-theta and 19.2°±0.2° 2-theta.

Embodiment 11: The crystalline form of Compound 1 according to embodiment 10, wherein the crystalline form exhibits further peaks in an x-ray powder diffraction (XRPD) pattern at 5.5°±0.2° 2-theta, 8.6°±0.2° 2-theta, 15.9°±0.2° 2-theta, 19.9°±0.2° 2-theta, and 24.1°±0.2° 2-theta.

Embodiment 12: The crystalline form of Compound 1 according to embodiment 11, wherein the crystalline form exhibits further peaks in an x-ray powder diffraction (XRPD) pattern at 10.6°±0.2° 2-theta, 11.0°±0.2° 2-theta, 15.4°±0.2° 2-theta, 21.0°±0.2° 2-theta, and 26.3°±0.2° 2-theta.

Embodiment 13: The crystalline form of Compound 1 according to any one of embodiments 9 to 12, wherein the crystalline form comprises a peak in a differential scanning calorimetry pattern of from about 205° C. to about 210° C.

Embodiment 14: The crystalline form of Compound 1 according to any one of embodiments 9 to 13, wherein the crystalline form exhibits a loss in mass in a thermal gravimetric analysis of less than about 2% upon heating the sample from about 25° C. to about 380° C.

Embodiment 15: The crystalline form of Compound 1 according to any one of embodiments 1 to 14, wherein the crystalline form exhibits less than about 10% degradation when the crystalline form is stored at 25° C. and 60% relative humidity for at least 7 days.

Embodiment 16: The crystalline form of Compound 1 according to any one of embodiments 1 to 15, wherein the crystalline form exhibits less than about 1% degradation when the crystalline form is stored at 25° C. and 60% relative humidity for at least 7 days.

Embodiment 17: The crystalline form of Compound 1 according to any one of embodiments 1 to 16, wherein the crystalline form exhibits less than about 10% degradation when the crystalline form is stored at 40° C. and 75% relative humidity for at least 7 days.

Embodiment 18: The crystalline form of Compound 1 according to embodiment 17, wherein the crystalline form exhibits less than about 1% degradation when the crystalline form is stored at 40° C. and 75% relative humidity for at least 7 days.

Embodiment 19: The crystalline form of Compound 1 according to any one of embodiments 1 to 18, wherein the crystalline form exhibits less than about 10% degradation when the crystalline form is stored at 60° C. for at least one week.

Embodiment 20: The crystalline form of Compound 1 according to embodiment 19, wherein the crystalline form exhibits less than about 1% degradation when the crystalline form is stored at 60° C. for at least one week.

Embodiment 21: The crystalline form of Compound 1 according to embodiment 1, wherein the crystalline form exhibits a peak in an x-ray powder diffraction (XRPD) pattern at 7.7°±0.2° 2-theta.

Embodiment 22: The crystalline form of Compound 1 according to embodiment 21, wherein the crystalline form exhibits further a peak in an x-ray powder diffraction (XRPD) pattern at 15.4°±0.2° 2-theta.

Embodiment 23: The crystalline form of Compound 1 according to embodiment 21 or embodiment 22, wherein the crystalline form exhibits further a peak in an x-ray powder diffraction (XRPD) pattern at 19.2°±0.2° 2-theta.

Embodiment 24: The crystalline form of Compound 1 according to any one of embodiments 21 to 23, wherein the crystalline form exhibits further a peak in an x-ray powder diffraction (XRPD) pattern at 13.7°±0.2° 2-theta.

Embodiment 25: The crystalline form of Compound 1 according to any one of embodiments 21 to 24, wherein the crystalline form exhibits further peaks in an x-ray powder diffraction (XRPD) pattern at 5.5°±0.2° 2-theta, 8.6°±0.2° 2-theta, 15.9°±0.2° 2-theta, 19.9°±0.2° 2-theta, and 24.1°±0.2° 2-theta.

Embodiment 26: The crystalline form of Compound 1 according to any one of embodiments 21 to 25, wherein the crystalline form exhibits further peaks in an x-ray powder diffraction (XRPD) pattern at 10.6°±0.2° 2-theta, 11.0°±0.2° 2-theta, 21.0°±0.2° 2-theta, and 26.3°±0.2° 2-theta.

Embodiment 27: A pharmaceutical composition comprising an amount of a crystalline form of Compound 1 according to any one of embodiments 1 to 26, and at least one pharmaceutically acceptable excipient.

Embodiment 28: A method of inhibiting PRC2 activity in a subject in need thereof, comprising administering to the subject an effective amount of a crystalline form of Compound 1 according to any one of embodiments 1 to 26.

Embodiment 29: The method according to embodiment 22, wherein the crystalline form of Compound 1 is the form of Compound 1 according to any one of embodiments 2 to 8.

Embodiment 30: The method according to embodiment 22, wherein the crystalline form of Compound 1 is the form of Compound 1 according to any one of embodiments 9 to 26.

Embodiment 31: A method of treating cancer in a subject, comprising administering to the subject a therapeutically effective amount of a crystalline form of Compound 1 according to any one of embodiments 1 to 26.

Embodiment 32: The method according to embodiment 31, wherein the crystalline form of Compound 1 is the form of Compound 1 according to any one of embodiments 2 to 8.

Embodiment 33: The method according to embodiment 31, wherein the crystalline form of Compound 1 is the form of Compound 1 of any one of embodiments 9 to 26.

Embodiment 34: A method of inhibiting PRC2 activity in a subject in need thereof, comprising administering to the subject an effective amount of the pharmaceutical composition according to embodiment 27.

Embodiment 35: A method of treating cancer in a subject, comprising administering to the subject an effective amount of a pharmaceutical composition according to embodiment 27.

Embodiment 36: The method according to any one of embodiments 31 to 33 and 35, wherein the cancer is selected from the group consisting of Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochondroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma.

Embodiment 37: The method according to any of embodiments 31 to 33, 35 and 36, wherein the cancer is a PRC2-associated cancer.

Embodiment 38: The method according to any one of embodiments 31 to 33, 35 and 36, wherein the cancer is prostate cancer, breast cancer, skin cancer, bladder cancer, liver cancer, pancreatic cancer, or head and neck cancer.

Embodiment 39: The method according to embodiment 38, wherein the cancer is prostate cancer.

Embodiment 40: The method according to embodiment 39, wherein the prostate cancer is metastatic prostate cancer, metastatic castration-resistant prostate cancer, localized high risk prostate cancer, recurrent prostate cancer, non-metastatic CRPC (nmCRPC), non-metastatic castration-sensitive prostate cancer, or metastatic castration-sensitive prostate cancer.

Embodiment 41: The method according to embodiment 38, wherein the cancer is breast cancer.

Embodiment 42: The method according to embodiment 38, wherein the cancer is skin cancer.

Embodiment 43: The method according to embodiment 38, wherein the cancer is bladder cancer.

Embodiment 44: The method according to embodiment 38, wherein the cancer is liver cancer.

Embodiment 45: The method according to embodiment 38, wherein the cancer is pancreatic cancer.

Embodiment 46: The method according to embodiment 38, wherein the cancer is head and neck cancer.

Embodiment 47: A method of treating cancer in a subject, comprising administering to the subject a therapeutically effective amount of a pharmaceutical composition according to embodiment 27.

Embodiment 48: The method according to embodiment 47, wherein the cancer is selected from the group consisting of Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochondroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma.

Embodiment 49: The method according to any of embodiments 47 and 48, wherein the cancer is a PRC2-associated cancer.

Embodiment 50: The method according to any one of embodiments 47 to 49, wherein the cancer is prostate cancer, breast cancer, skin cancer, bladder cancer, liver cancer, pancreatic cancer, or head and neck cancer.

Embodiment 51: The method according to embodiment 50, wherein the cancer is prostate cancer.

Embodiment 52: The method according to embodiment 51, wherein the prostate cancer is metastatic prostate cancer, metastatic castration-resistant prostate cancer, localized high risk prostate cancer, recurrent prostate cancer, non-metastatic CRPC (nmCRPC), non-metastatic castration-sensitive prostate cancer, or metastatic castration-sensitive prostate cancer.

Embodiment 53: The method according to embodiment 50, wherein the cancer is breast cancer.

Embodiment 54: The method according to embodiment 50, wherein the cancer is skin cancer.

Embodiment 55: The method according to embodiment 50, wherein the cancer is bladder cancer.

Embodiment 56: The method according to embodiment 50, wherein the cancer is liver cancer.

Embodiment 57: The method according to embodiment 50, wherein the cancer is pancreatic cancer.

Embodiment 58: The method according to embodiment 50, wherein the cancer is head and neck cancer.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A crystalline form of Compound 1:

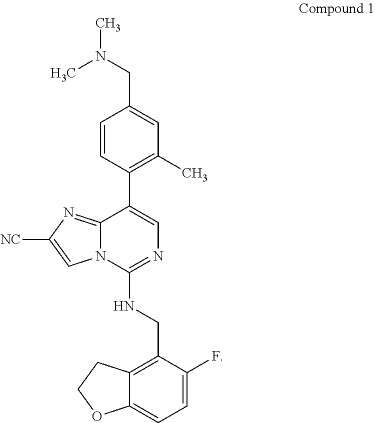 [SMILES: Cc1cc(CN(C)C)ccc1-c1cnc(NCc2c(F)ccc3c2CCO3)n2cc(C#N)nc12]

2. The crystalline form of Compound 1 according to claim 1, wherein the crystalline form exhibits a peak in an x-ray powder diffraction (XRPD) pattern at 8.1°±0.2° 2-theta.

3. The crystalline form of Compound 1 according to claim 2, wherein the crystalline form exhibits a further peak in an x-ray powder diffraction (XRPD) pattern at 9.6°±0.2° 2-theta.

4. The crystalline form of Compound 1 according to claim 3, wherein the crystalline form exhibits further peaks in an x-ray powder diffraction (XRPD) pattern at 5.7°±0.2° 2-theta, 19.7°±0.2° 2-theta, and 22.0°±0.2° 2-theta.

5. The crystalline form of Compound 1 according to claim 4, wherein the crystalline form exhibits further peaks in an x-ray powder diffraction (XRPD) pattern at 9.8°±0.2° 2-theta, 15.2°±0.2° 2-theta, and 17.7°±0.2° 2-theta.

6. The crystalline form of Compound 1 according to claim 1, wherein the crystalline form exhibits a peak in an x-ray powder diffraction (XRPD) pattern at 7.7°±0.2° 2-theta.

7. The crystalline form of Compound 1 according to claim 6, wherein the crystalline form exhibits further peaks in an x-ray powder diffraction (XRPD) pattern at 13.7°±0.2° 2-theta and 19.2°±0.2° 2-theta.

8. The crystalline form of Compound 1 according to claim 7, wherein the crystalline form exhibits further peaks in an x-ray powder diffraction (XRPD) pattern at 5.5°±0.2° 2-theta, 8.6°±0.2° 2-theta, 15.9°±0.2° 2-theta, 19.9°±0.2° 2-theta, and 24.1°±0.2° 2-theta.

9. The crystalline form of Compound 1 according to claim 8, wherein the crystalline form exhibits further peaks in an x-ray powder diffraction (XRPD) pattern at 10.6°±0.2° 2-theta, 11.0°±0.2° 2-theta, 15.4°±0.2° 2-theta, 21.0°±0.2° 2-theta, and 26.3°±0.2° 2-theta.

10. The crystalline form of Compound 1 according to any one of claims 1 to 9, wherein the crystalline form exhibits less than about 10% degradation when the crystalline form is stored at 25° C. and 60% relative humidity for at least 7 days.

11. The crystalline form of Compound 1 according to any one of claims 1 to 9, wherein the crystalline form exhibits less than about 10% degradation when the crystalline form is stored at 40° C. and 75% relative humidity for at least 7 days.

12. A pharmaceutical composition comprising an amount of a crystalline form of Compound 1 according to any one of claims 1 to 11, and at least one pharmaceutically acceptable excipient.

13. A method of inhibiting PRC2 activity in a subject in need thereof, comprising administering to the subject an effective amount of a crystalline form of Compound 1 according to any one of claims 1 to 11.

14. A method of treating cancer in a subject, comprising administering to the subject a therapeutically effective amount of a crystalline form of Compound 1 according to any one of claims 1 to 11.

15. A method of treating cancer in a subject, comprising administering to the subject an effective amount of a pharmaceutical composition according to claim 12.

16. The method according to claim 14 or 15, wherein the cancer is prostate cancer, breast cancer, skin cancer, bladder cancer, liver cancer, pancreatic cancer, or head and neck cancer.

17. The method according to claim 16, wherein the cancer is prostate cancer.

18. The method according to claim 17, wherein the prostate cancer is metastatic prostate cancer, metastatic castration-resistant prostate cancer, localized high risk prostate cancer, recurrent prostate cancer, non-metastatic CRPC (nmCRPC), non-metastatic castration-sensitive prostate cancer, or metastatic castration-sensitive prostate cancer.
